(19) United States
(12) Patent Application Publication
(10) Pub. No.: US 2021/0212962 A1
(43) Pub. Date: Jul. 15, 2021

(54) METHODS AND COMPOSITIONS FOR THE TREATMENT OF GRANULOMA ANNULARE OR SUBCUTANEOUS INFLAMMATION AND NON-INFECTION GRANULOMATOUS DISEASES

(71) Applicant: Sol-Gel Technologies Ltd., Ness Ziona (IL)

(72) Inventors: Marcel Zighelboim, Kiryat Motzkin (IL); Hila Hakak Djerbi, Tel Aviv (IL); Karine Neimann, Ness Ziona (IL)

(73) Assignee: Sol-Gel Technologies Ltd., Ness Ziona (IL)

(21) Appl. No.: 17/147,602

(22) Filed: Jan. 13, 2021

Related U.S. Application Data
(60) Provisional application No. 62/960,164, filed on Jan. 13, 2020.

(51) Int. Cl. A61K 31/05; A61K 31/44; A61K 9/00; A61K 9/06; A61P 17/00

(52) U.S. Cl. CPC A61K 31/05; A61K 31/44; A61P 17/00; A61K 9/06; A61K 9/0014

(57) ABSTRACT

The present invention, in some embodiments thereof, relates to topical compositions and methods of treatment, prevention and/or amelioration of granuloma annulare, a non-infection granulomatous disease, or a subcutaneous inflammation by topical or intralesional administration of compositions comprising tapinarof.

### FIELD OF THE INVENTION

The present invention, in some embodiments thereof, relates to topical compositions and methods of treatment, prevention and/or amelioration of granuloma annulare, and non-infection granulomatous diseases, ocular sarcoidosis or a subcutaneous inflammation, the method comprises topical or intralesional administration of compositions comprising tapinarof.

### BACKGROUND OF THE INVENTION

Granuloma annulare (GA) is a cutaneous granulomatous disease. It is not caused by an infection and is the most common non-infectious granulomatous disease. The disease is benign and often self-limited. Granuloma annulare usually presents as erythematous plaques or papules arranged in an annular configuration on the upper extremities. Despite being a benign disease, it can be associated with more serious conditions such as HIV or malignancy. Granuloma annulare affects patients of all ages. The prevalence of granuloma annulare is estimated to be 0.1% to 0.4% and the estimated incidence is 0.1% to 0.4%. Incidence is highest in women, with a ratio of 2.3 to 1.0 over men. Approximately 15 percent of all patients with granuloma annulare will have more than 10 lesions (i.e., disseminated granuloma annulare).

Although numerous theories have been proposed to explain the cause of granuloma annulare, the pathogenesis of this cutaneous disease remains unclear. It has been reported to follow trauma, malignancy, viral infections (including human immunodeficiency virus [HIV], Epstein-Barr virus, and herpes zoster), insect bites, and tuberculosis skin tests. [Smith M D, Downie J B, DiCostanzo D. Granuloma annulare. Int J Dermatol. 1997; 36: 326-33; Prendiville J S. Chapter 44. Granuloma Annulare. In: Goldsmith L A, Katz S I, Gilchrest B A, Paller A S, Leffell D J, Wolff K. eds. Fitzpatrick's Dermatology in General Medicine, 8e New York, N.Y.: McGraw-Hill; 2012; Samlaska C P, Sandberg G D, Maggio K L, et al. Generalized perforating granuloma annulare, J Am Acad Dermatol 1992; 27: 319-322].

Patients with diabetes mellitus had a higher incidence of chronic relapsing granuloma annulare than patients without diabetes.

Some isolated cases of granuloma annulare found in association with malignant neoplasm have been reported. [Indian Dermatol Online J. 2018 March-April; 9(2): 117-119]

Granuloma annulare has been described as a paraneoplastic granulomatous reaction to solid organ tumors, Hodgkin disease, non-Hodgkin lymphoma and granulomatous mycosis fungoides. In these patients, the clinical pattern is frequently atypical, with painful lesions in unusual locations, including the palms and soles. [Aaron R. Mangold et al., J Am Acad Dermatol. 2018 79(5):913-920; doi: 10.1016/j.jaad.2018.06.022. Epub 2018 Jun. 18; Li A, Hogan D J, Sanusi I D, Smoller B R. Granuloma annulare and malignant neoplasms, Am J Dermatopathol. 2003, 25(2): 113-116; Harman R R. Hodgkin's disease, seminoma of testicle and widespread granuloma annulare. Br J Dermatol. 1977; 97(Suppl 15): 50-51.] Many reports supporting or refuting the association of granuloma annulare with diabetes mellitus have been published. [Alirezaei P, Farshchian M. Granuloma annulare: relationship to diabetes mellitus, thyroid disorders and tuberculin skin test. Clin Cosmet Investig Dermatol. 2017 10:141-145; Gerald S Kidd et al. Glucose Tolerance in Granuloma Annulare Diabetes Care 1985 8(4): 380-384; Susannah M C George, Shernaz Walton, Granuloma annulare Br J Diabetes 2016, 16:58-61; Richard Romaine et al. Papular Granuloma Annulare and Diabetes Mellitus, Arch Dermatol. 1968, 98(2): 152-154.]

In a retrospective study of 84 patients, 12% were found to have diabetes mellitus.

Granuloma annulare lesions are approximately distributed as follows: 60% isolated to the hands and arms; 20% on the legs and feet; 7% on both upper and lower extremities and 5% on the trunk plus other areas.

The four main clinical variants of granuloma annulare are:

Localized

Disseminated

Subcutaneous; and

Perforating.

The localized form of granuloma annulare composes 75 percent of cases. Localized granuloma annulare starts as a ring of small, firm, flesh-colored or red papules.

Most cases of localized granuloma annulare are diagnosed in patients before 30 years of age. As the condition progresses, there is some central involution, and the ring of papules slowly increases from 0.5 to 5.0 cm in diameter. The lesions may be isolated or coalesce into plaques. They are found on the lateral or dorsal surfaces of the hands and feet.

The Disseminated or generalized granuloma annulare is similar to the localized variant but is more widespread, having 10 or more lesions. The papules may fuse to form annular lesions on the extremities, trunk, and neck. In contrast to the localized form, these lesions may persist for three to four years or longer.

The Subcutaneous granuloma annulare is diagnosed primarily in children two to five years of age. The lesions are asymptomatic, rapidly growing subcutaneous nodules on the extremities, hands, scalp, buttocks, and pretibial and periorbital areas.

The Perforating granuloma annulare is rare and occurs most often in children and young adults. It is also more common in women. Perforating granuloma annulare can have localized and generalized forms.

The localized form is found on the upper limbs and pelvis, and the generalized form, which is more common, is present on the abdominal area, trunk, and upper and lower limbs.

25% of patients report pruritus, and 25% report pain, mainly in lesions located on the palms.

The following local treatments are used for treating granuloma annulare: topical corticosteroid ointment under occlusion, intralesional steroid injections, destruction by cryotherapy or laser ablation, imiquimod cream or topical calcineurin inhibitors (tacrolimus and pimecrolimus).

The following systemic treatments are used for treating granuloma annulare: systemic steroids, osotretinoin, methotrexate, potassium iodide, pentoxifylline, dapsone, hydroxychloroquine, allopurinol (note: allopurinol has also been cited as a cause of disseminated granuloma annulare), photochemotherapy (PUVA), combination of antibiotics (once monthly) such as: rifampicin, ofloxacin, minocycline.

Currently there is no efficient treatment available for subcutaneous GA. In the treatment by surgical excision, the GA recurs.

Granuloma annulare lesions typically persist for 3 to 4 years, and following spontaneous resolution of lesions, relapse may occur. There is no standard treatment regimen or approach employed in the management of patients with granuloma annulare. Furthermore, the response to long-term conventional treatment is generally poor.

A subcutaneous inflammation occurs when the subcutaneous tissue (which holds and supports together with the dermis the epidermis) is injured. If the tissue is injured, the entire structure of the skin may be greatly affected, even though the surface of the skin itself might show only minor changes. Inflammation of subcutaneous adipose tissue (the fatty layer under the skin—panniculus adiposus) clinically appears as a deep seated inflammatory nodule. Fatty tissue reacts to injury in a characteristic sequence. The site of injury is first infiltrated by neutrophils, followed by lymphocytes, histiocytes, and eventually fibroblasts.

Inflammatory diseases of the subcutaneous fat are grouped under the general term “panniculitis”. Many diseases cause panniculitis, but the clinical manifestation is often quite similar. Inflammation in the fatty tissues causes pain, tenderness, ill-defined erythematous nodules, and occasionally ulceration of the overlying skin.

Thus, there is a need for a better method of treating granuloma annulare and for the treatment of subcutaneous inflammation. The present invention satisfies this need and provides other benefits that will be apparent from the disclosure below.

### SUMMARY OF THE INVENTION

In some embodiments, this invention provides a method of treatment, prevention or alleviation of granuloma annulare, cutaneous sarcoidosis, a non-infection granulomatous disease, or a subcutaneous inflammation, comprising administering to a subject in need thereof a therapeutically effective amount of a topical or intralesional composition comprising from about 0.25% w/w to about 10.0% w/w tapinarof and a carrier.

In other embodiments, the composition further comprises a PDE4 inhibitor. In other embodiments, the PDE4 inhibitor is selected from the group consisting of roflumilast, apremilast, crisaborole, rolipram, cilomilast, ibudilast, piclamilast and combinations thereof. In other embodiments, the PDE4 inhibitor is roflumilast. In other embodiments, the composition comprises from about 0.25% w/w to about 10.0% w/w tapinarof and from about 0.25% w/w to about 3% w/w at least one PDE4 inhibitor.

In some embodiments, this invention provides a method of treatment, prevention or alleviation of granuloma annulare, a non-infection granulomatous disease, or a subcutaneous inflammation, comprising administering to a subject in need thereof a therapeutically effective amount of a topical or intralesional a first composition comprising from about 0.25% w/w to about 10.0% w/w tapinarof and a second composition comprising from about 0.25% w/w to about 3% w/w PDE4 inhibitor, wherein the first composition and second composition are administered concurrently or sequentially, at least once a day until remission. In other embodiments, the PDE4 inhibitor is selected from the group consisting of roflumilast, apremilast, crisaborole, rolipram, cilomilast, ibudilast, piclamilast and combinations thereof. In other embodiments, the PDE4 inhibitor is roflumilast.

In some embodiments this invention provides a dosage form for the treatment, prevention and/or amelioration of granuloma annulare, a non-infection granulomatous disease, or a subcutaneous inflammation, wherein the dosage form comprising a therapeutically effective amount of a topical or intralesional composition comprising from about 0.25% w/w to about 10.0% w/w tapinarof and a carrier, wherein the composition is formulated in a dosage form selected from a cream, a lotion, a gel, an ointment, an emulsion, a solution, a suspension, an elixir, a tincture, a paste, a foam, an aerosol, a spray, a patch, a transdermal patch, via roll-on applicator, microneedles and an applicator syringe.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIGS. 1A-1C present results of Generalized Granuloma Annulare treatment following two weeks of treatment, twice a day, using 1% tapinarof lotion (Examples 1 and 2). The pictures demonstrate a clear improvement.

FIGS. 2A-2B present a rebound effect following the treatment presented in FIG. 1, with erythema, pruritus and folliculitis following 4 weeks of treatment, which was treated with an anti-inflammatory agent (Elocom), demonstrating a clear improvement following 5 days of treatment with an anti-inflammatory agent.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

The compositions, dosage forms, kits, regimens and methods of this invention are suitable for the treatment, prevention or alleviation of granuloma annulare, non-infection granulomatous diseases or subcutaneous inflammation.

### Compositions

In some embodiments, this invention provides a topical or intralesional composition for the treatment, prevention and/or amelioration of granuloma annulare, non-infection granulomatous diseases (such as cutaneous sarcoidosis, ocular sarcoidosis) or a subcutaneous inflammation, comprising from about 0.25% w/w to about 10.0% w/w tapinarof and a carrier. In one embodiment, the topical or intralesional composition is for the treatment, prevention and/or amelioration of granuloma annulare. In another embodiment, the topical or intralesional composition is for the treatment, prevention and/or amelioration of localized granuloma annulare. In another embodiment, the topical or intralesional composition is for the treatment, prevention and/or amelioration of disseminated granuloma annulare. In another embodiment, the topical or intralesional composition is for the treatment, prevention and/or amelioration of subcutaneous granuloma annulare. In another embodiment, the topical or intralesional composition is for the treatment, prevention and/or amelioration of perforating granuloma annulare. In another embodiment, the topical or intralesional composition is for the treatment, prevention and/or amelioration of subcutaneous inflammation. In another embodiment, the topical or intralesional composition is for the treatment, prevention and/or amelioration of both granuloma annulare and subcutaneous inflammation. In another embodiment, the topical or intralesional composition is for the treatment, prevention and/or amelioration of cutaneous sarcoidosis. In another embodiment, the topical or intralesional composition is for the treatment, prevention and/or amelioration of ocular sarcoidosis.

In another embodiment, the tapinarof has a purity greater than 97%. In another embodiment, a purity of between 97% to 100%. In another embodiment, a purity of between 98% to 100%. In another embodiment 97%, 98%, 99% or greater than 99%.

In one embodiment, the composition for the treatment, prevention and/or amelioration of granuloma annulare, a non-infection granulomatous disease, or a subcutaneous inflammation comprises between 0.25% w/w to about 10.0% w/w tapinarof. In another embodiment, the composition comprises between about 0.25% w/w to about 3% w/w tapinarof. In another embodiment, the composition comprises between about 0.25% w/w to about 5% w/w tapinarof. In another embodiment, between about 1% w/w to about 10% w/w tapinarof. In another embodiment, between about 1% w/w to about 5% w/w tapinarof. In another embodiment, between about 2% w/w to about 10% w/w tapinarof. In another embodiment, between about 2% w/w to about 7% w/w tapinarof. Exemplary dosages, strengths and concentrations of tapinarof in the topical or intralesional compositions, are in the range of from about or at 0.25%, 0.5%, 1%, 2%, 3%, 4%, 5%, 6%, 7%, 8%, 9% or 10% w/w. Typical strengths in the topical compositions of this invention are 1%, 2% or 5% w/w tapinarof. In another embodiment, the non-infection granulomatous disease include cutaneous sarcoidosis, ocular sarcoidosis.

Tapinarof (3,5-dihydroxy-4-isopropyl-trans-stilbene) is a pharmaceutical active agent investigated for the treatment of atopic dermatitis, psoriasis and psoriatic disorders (Zang Y N, et al., Int J Clin Pharmacol Ther. 2016 February; 54(2):87-95). The 3,5-dihydroxy-4-isopropyl-stilbene is also known as benvitimod.

Tapinarof is a first-in-class drug, whose mechanism is not yet fully understood.

In another embodiment, the composition comprises 1% or 2% w/w tapinarof.

In one embodiment, the composition for the treatment, prevention and/or amelioration of granuloma annulare, a non-infection granulomatous disease, or a subcutaneous inflammation, further comprises a PDE4 inhibitor. In another embodiment, the non-infection granulomatous disease includes cutaneous sarcoidosis or ocular sarcoidosis.

In other embodiments, the PDE4 inhibitor is selected from the group consisting of roflumilast, apremilast, crisaborole, rolipram, cilomilast, ibudilast, piclamilast and combinations thereof.

In some embodiments, there is provided a topical composition comprising from about 0.25% w/w to about 10.0% w/w tapinarof, from about 0.25% w/w to about 3% w/w of a PDE4 inhibitor and a carrier suitable for topical or intralesional administration. In some embodiments, there is provided a topical composition comprising from about 0.25% w/w to about 5% w/w tapinarof, from about 0.25% w/w to about 3% w/w of a PDE4 inhibitor and a carrier suitable for topical or intralesional administration.

PDE4 inhibitors (phosphodiesterase Type 4 inhibitors) are blocking the degradation of cyclic adenosine monophosphate (cAMP) by phosphodiesterase 4 (PDE4).

PDE4 inhibitors have been investigated for a number of medical indications, like CNS disorders, chronic obstructive pulmonary disease (COPD), asthma and rheumatoid arthritis.

The systemic administration of PDE4 inhibitors is accompanied by emesis, which is a big drawback. The topical administration of PDE4 inhibitor comprising combination drugs provided in this disclosure avoids said systemic emetic effect.

The PDE4 inhibitor in the composition of this disclosure is selected from roflumilast, apremilast, crisaborole, rolipram. cilomilast, ibudilast, piclamilast and combinations thereof.

Without wishing to be bound by theory, the combination of tapinarof with a PDE4 inhibitor and optionally with an additional active agent provides additive and/or synergistic effects, enabling lower concentrations and dosages of the active agents, thus minimizing their side-effects. Both tapinarof and the PDE4 inhibitor have anti-inflammatory and anti-oxidant activities and their combinations have better activity than each of its constituents. “Phosphodiesterase-4 (PDE4) is the major enzyme class responsible for the hydrolysis of cyclic adenosine monophosphate (cAMP), an intracellular second messenger that controls a network of proinflammatory and anti-inflammatory mediators” (Textbook of Pediatric Rheumatology (Seventh Edition), 2016, Elsevier).

The frequency of administration of the composition of this invention can be determined empirically. Exemplary frequencies are once daily, twice daily, weekly, bi-weekly or monthly. Typical administration frequencies of the topical or intralesional compositions of this invention are once daily and twice daily.

In another embodiment, the composition for the treatment, prevention and/or amelioration of granuloma annulare, a non-infection granulomatous disease (such as cutaneous sarcoidosis, ocular sarcoidosis), or a subcutaneous inflammation is administered for a period between one week to six weeks, up to six months or until remission. In another embodiment, the composition is administered for a period between one week to six weeks. In another embodiment, the composition is administered for a period between two weeks to six weeks. In another embodiment, the composition is administered for a period between two weeks to four weeks. In some embodiments, the composition is administered for a period longer than six weeks. In some embodiments, the composition is administered for a period of up to six months. In some embodiments, the composition is administered until remission.

Pharmaceutical carriers or vehicles suitable for preparation of the compositions provided herein include any such carriers known to those skilled in the art to be suitable for the particular mode of administration.

In some embodiment, the composition for the treatment, prevention and/or amelioration of granuloma annulare, a non-infection granulomatous disease, or a subcutaneous inflammation comprises between 0.25% w/w to about 10% w/w tapinarof as the active ingredient and a carrier, a solvent, an emollient, a surfactant, a chelating agent, a gelling agent, a wetting agent, a penetration enhancer, a preservative, an anti-oxidant, a buffer or any combination thereof. Each represents a separate embodiment of this invention. In another embodiment, the non-infection granulomatous disease includes cutaneous sarcoidosis or ocular sarcoidosis.

In some embodiment, the composition for the treatment, prevention and/or amelioration of granuloma annulare, a non-infection granulomatous disease, or a subcutaneous inflammation comprises between 0.25% w/w to about 3% w/w roflumilast as the active ingredient (second composition) and a carrier, wherein this second composition is administered in combination with a composition comprising between 0.25% w/w to about 10% w/w tapinarof as the active ingredient (first composition) and a carrier. In another embodiment, the non-infection granulomatous disease includes cutaneous sarcoidosis or ocular sarcoidosis.

In some embodiment, the composition for the treatment, prevention and/or amelioration of granuloma annulare, a non-infection granulomatous disease, or a subcutaneous inflammation comprises a combination of between 0.25% w/w to about 10% w/w tapinarof and between 0.25% w/w to about 3% w/w roflumilast and a carrier, a solvent, an emollient, a surfactant, a chelating agent, a gelling agent, a wetting agent, a penetration enhancer, a preservative, an anti-oxidant, a buffer, or any combination thereof. In another embodiment, the non-infection granulomatous disease includes cutaneous sarcoidosis or ocular sarcoidosis.

In some embodiments, the compositions of this invention comprise a carrier. In another embodiment, the carrier is water.

In some embodiments, the compositions of this invention comprise a penetration enhancer. In another embodiment, the penetration enhancer is selected from the group consisting of dimethylsulfoxide (DMSO), diethylene glycol monoethyl ether (Transcutol), methylsulfonylmethane (MSM), oleic acid, oleyl alcohol, propylene glycol, dimethyl isosorbide, isopropyl myristate, diethyl sebacate, ethanol, isopropyl alcohol, a polyethylene glycol, hexylene glycol, glycofurol and any combination thereof. Each represent a separate embodiment of this invention. In another embodiment, the penetration enhancer is in amount of between about 10% w/w to about 25% w/w. In another embodiment, the penetration enhancer is in amount of between about 10% w/w to about 15% w/w. In another embodiment, the penetration enhancer is in amount of between about 15% w/w to about 25% w/w.

In some embodiments, the compositions of this invention comprise an emollient. In another embodiment, the emollient is selected from the group consisting of castor oil, mineral oil, vegetable oil, soybean oil, shea butter, cocoa butter, paraffin, beeswax, oleic acid, squalene, cetyl alcohol, isopropyl myristate, urea, glycerol, propylene glycol, lactic acid and any combination thereof. Each represent a separate embodiment of this invention. In another embodiment, the emollient is in amount of between about 3% w/w to about 10% w/w. In another embodiment, the emollient is in amount of between about 5% w/w to about 10% w/w.

In some embodiments, the compositions of this invention comprise a chelating agent. In another embodiment, the chelating agent is selected from the group consisting of EDTA deferoxamine, deferiprone, deferasirox, dimercaptosuccinic acid (succimer) triethylenetetramine (trientine) and any combination thereof. Each represent a separate embodiment of this invention. In another embodiment, the chelating agent is in amount of between about 0.05% w/w to about 0.5% w/w. In another embodiment, the chelating agent is in amount of between about 0.05% w/w to about 0.2% w/w.

In some embodiments, the compositions of this invention comprise an anti-oxidant. In another embodiment, the anti-oxidant is selected from the group consisting of butylated hydroxytoluene (BHT), parabens, propyl gallate, ascorbic acid, flavones, flavanones, flavonols, stilbenoids, gallic acid, cinnamic acid, ellagic acid, salicylic acid, curcumin, eugenol, citric acid and any combination thereof. Each represent a separate embodiment of this invention. In another embodiment, the anti-oxidant is in amount of between about 0.05% w/w to about 0.5% w/w. In another embodiment, the anti-oxidant is in amount of between about 0.1% w/w to about 0.5% w/w. In another embodiment, the anti-oxidant is in amount of between about 0.1% w/w to about 0.3% w/w.

In some embodiments, the compositions of this invention comprise a solvent. In another embodiment the solvent is selected from the group consisting of propylene glycol, diethylene glycol monoethyl ether (Transcutol), dimethylsulfoxide (DMSO), diethyl sebacate, polyethylene glycol, oleyl alcohol, isosorbide dimethyl ether, ethanol, isopropyl alcohol, isopropyl myristate, oleic acid, hexylene glycol, glycerin, glycofurol and any combination thereof. Each represent a separate embodiment of this invention. In another embodiment, the solvent is in amount of between about 5 w/w to about 25% w/w. In another embodiment, the solvent is in amount of between about 5% w/w to about 20% w/w. In another embodiment, the solvent is in amount of between about 10% w/w to about 15% w/w. In another embodiment, the solvent is in amount of between about 10% w/w to about 20% w/w.

In some embodiments, the compositions of this invention comprise a preservative. In another embodiment the preservative is selected from the group consisting of benzoic acid, methylparaben, chlorocresol, Phenoxyethanol, propyl paraben, benzalkonium chloride, benzyl alcohol, imidazolidinyl urea, sodium benzoate, sorbic acid, thimerosal sorbic acid, sodium sorbate, sodium sulfite, ethanol, tocopherols and any combination thereof. Each represent a separate embodiment of this invention. In another embodiment, the preservative is in amount of between about 0.10 w/w to about 1% w/w. In another embodiment, the preservative is in amount of between about 0.15% w/w to about 0.50% w/w. In another embodiment, the preservative is in amount of between about 0.25% w/w to about 0.80% w/w.

In some embodiments, the compositions of this invention comprise a buffer. In another embodiment, the buffer is selected from the group consisting of citric acid, phosphoric acid, acetic acid, tartaric acid, glutamic acid, aspartic acid, malic acid, succinic acid, fumaric acid, salt thereof, and any combination thereof. Each represent a separate embodiment of this invention. In another embodiment, the buffer is in amount of between about 0.05 w/w to about 0.5% w/w. In another embodiment, the buffer is in amount of between about 0.05% w/w to about 0.30% w/w. In another embodiment, the buffer is between about 0.1% w/w to about 0.40% w/w.

In some embodiments, the compositions of this invention comprise a gelling agent. In another embodiment, the gelling agent is selected from the group consisting of carbomer homopolymer type A (Carbopol®981, Carbopol®980), Sepineo P600, Natrosol hydroxyethylcellulose, starch, pectin, gelatin, agar, alginic acid and salts thereof, Carbomer® 934, carboxymethylcellulose, hydroxypropylmethylcellulose and any combination thereof. Each represent a separate embodiment of this invention. In another embodiment, the gelling agent is in amount of between about 0.5 w/w to about 2% w/w. In another embodiment, the gelling agent is in amount of between about 0.5% w/w to about 1.5% w/w. In another embodiment, the gelling agent is in amount of between about 0.5% w/w to about 1% w/w.

In some embodiments, the compositions of this invention comprise a surfactant. In another embodiment, the surfactant is selected from the group consisting of carbomer copolymer type B (Pemulen®TR-1), sorbitan monooleate (Span80), polysorbate 80 (Tween 80), polysorbate 20, polysorbate 60 (Tween 60), sodium dodecylsulfate, cetearyl alcohol, dioctyl sodium sulfosuccinate, glyceryl oleate, glyceryl stearate, poloxamers and any combination thereof. Each represent a separate embodiment of this invention. In another embodiment, the surfactant is in amount of between about 0.1 w/w to about 6% w/w. In another embodiment, the surfactant is in amount of between about 0.1% w/w to about 0.3% w/w. In another embodiment, the surfactant is in amount of between about 0.1% w/w to about 1% w/w. In another embodiment, the surfactant is in amount of between about 1% w/w to about 3% w/w. In another embodiment, the surfactant is in amount of between about 2% w/w to about 6% w/w. In another embodiment, the surfactant is in amount of between about 1% w/w to about 2% w/w.

In some embodiments, the compositions of this invention comprise a wetting agent. In another embodiment, the wetting agent is selected from the group consisting of PEG 400, glycerin, poloxamers, sorbitan monooleate (Span80), polysorbate 80 (Tween 80), polysorbate 20, polysorbate 60 (Tween 60), benzalkonium chloride, sodium dodecylsulfate and any combination thereof. Each represent a separate embodiment of this invention. In another embodiment, the wetting agent is in amount of between about 3 w/w to about 10% w/w. In another embodiment, the wetting agent is in amount of between about 4% w/w to about 6% w/w. In another embodiment, the wetting agent is in amount of between about 3% w/w to about 6% w/w. In another embodiment, the wetting agent is in amount of 5% w/w.

In some embodiments, the compositions of this invention are formulated as a cream, a lotion, a gel, an ointment, an emulsion, a solution, a suspension, an elixir, a tincture, a paste, a foam, an aerosol, a spray, a patch, a transdermal patch, via roll-on applicator, microneedles, an applicator syringe. or any other formulation suitable for topical administration. The preferred compositions are the cream, the gel, the foam and the lotion.

In some embodiments, the composition of this invention is a gel. In another embodiment, the gel composition comprises a surfactant, a solvent, a wetting agent, a gelling agent, a penetration enhancer, a preservative and an anti-oxidant. In another embodiment, the surfactant, the solvent, the wetting agent, the gelling agent, the penetration enhancer, the preservative and the anti-oxidant are described above.

In some embodiment the composition is a gel comprising between 0.25% w/w to about 5% w/w tapinarof. In some embodiment the composition is a gel comprising 1%, 2% or 5% w/w tapinarof.

In some embodiments, the gel composition comprises between 2-6% w/w a surfactant, between 3-6% a wetting agent, between 0.1-1% a preservative, between 0.5-2% w/w a gelling agent, between 10-20% w/w a solvent, between 10-25% w/w a penetration enhancer and between 0.1-0.5 w/w an anti-oxidant.

In some embodiments, the gel composition comprises 1%, 2% w/w tapinarof, 2%, 3%, 4% 5% or 6% w/w a surfactant, 3%, 4%, 5% or 6% a wetting agent, 0.1%, 0.2%, 0.3%, 0.4%, 0.5%, 0.6%, 0.7%, 0.8%, 0.9%, 1% a preservative, 0.5%, 0.7%, 1%, 1.1%, 1.2%, 1.3%, 1.4%, 1.5%, 1.6%, 1.7%, 1.9% or 2% w/w a gelling agent, 10%, 11%, 12%, 13%, 14%, 15%, 16%, 17%, 18%, 19% or 20% w/w a solvent, 10%, 12%, 14%, 16%, 17%, 18%, 19%, 20%, 21%, 22%, 23%, 24%, 25% w/w a penetration enhancer and 0.1%, 0.2%, 0.3%, 0.4%, 0.5% w/w an anti-oxidant.

In some embodiments, the composition of this invention is a lotion. In another embodiment, the lotion comprises a surfactant, an emollient, a solvent, a gelling agent, a penetration enhancer, a chelating agent, a preservative, a buffer and an anti-oxidant. In another embodiment, the surfactant, the emollient, the solvent, the gelling agent, the penetration enhancer, the chelating agent, the preservative, the buffer and the anti-oxidant are described above.

In some embodiment the composition is a lotion comprising between 0.25% w/w to about 5% w/w tapinarof. In some embodiment the composition is a lotion comprising 1%, 2% or 5% w/w tapinarof. In some embodiment the composition is a lotion comprising 1% w/w tapinarof.

In another embodiment, the lotion comprises between 0.1% w/w to 1% w/w a surfactant, between 3% w/w to 10% w/w an emollient, between 5% w/w to 25 w/w solvent, between 0.5% w/w to 2% w/w a gelling agent, between 10% w/w to 25% w/w a penetration enhancer, between 0.05% w/w to 5% w/w a chelating agent, between 0.1% to 1% w/w a preservative, between 0.05% w/w to 0.5% w/w a buffer and between 0.05% w/w to 0.5% w/w an anti-oxidant. In another embodiment, the surfactant, the emollient, the solvent, the gelling agent, the penetration enhancer, the chelating agent, the preservative, the buffer and the anti-oxidant are described above.

In some embodiments the compositions are described in Examples 2-5.

In one embodiment, the composition is a tapinarof lotion comprising 1% w/w tapinarof (Example 2).

In one embodiment, the composition is a tapinarof-hydrogel comprising 2% w/w tapinarof (Example 3).

In one embodiment, the composition is a tapinarof-hydrogel comprising 2% w/w tapinarof (Example 4).

In one embodiment, the composition is a tapinarof-hydrogel comprising 2% w/w tapinarof (Example 5).

### Dosage Forms

In some embodiments, this invention provides a dosage form for the treatment, prevention and/or amelioration of granuloma annulare, a non-infection granulomatous disease, or a subcutaneous inflammation, the dosage form comprises the composition of this invention, wherein the composition is formulated in a dosage form selected from a cream, a lotion, a gel, an ointment, an emulsion, a solution, a suspension, an elixir, a tincture, a paste, a foam, an aerosol, a spray, a patch, a transdermal patch, via roll-on applicator microneedles and an applicator syringe. Each represents a separate embodiment of this invention. In another embodiment, the dosage form is suitable for topical or intralesional administration. In another embodiment, the non-infection granulomatous disease includes cutaneous sarcoidosis or ocular sarcoidosis.

In another embodiment, the dosage form is a gel. In another embodiment, the dosage form is a cream. In another embodiment, the dosage form is a lotion. In another embodiment, the dosage form is a foam.

In another embodiment, the dosage form is administered at least once daily. In another embodiment, the dosage form is administered once or twice per day until remission. In another embodiment, the dosage form is administered for a period of between two weeks to six weeks, up to six months or until remission. In another embodiment, the dosage form is administered for a period of between two weeks to six weeks. In another embodiment, the dosage form is administered for a period of between two weeks to four weeks. In another embodiment, the dosage form is administered for a period of between one week to four weeks. In some embodiments, the composition is administered for a period longer than six weeks. In some embodiments, the composition is administered for a period of up to six months. In some embodiments, the composition is administered until remission.

In some embodiments, this inventions provides a dosage form for the treatment, prevention and/or amelioration of granuloma annulare, a non-infection granulomatous disease, or a subcutaneous inflammation, wherein the dosage form comprises a therapeutically effective amount of a topical or intralesional composition comprising from about 0.25% w/w to about 10.0% w/w tapinarof and a carrier, wherein the composition is formulated in a dosage form selected a cream, a lotion, a gel, an ointment, an emulsion, a solution, a suspension, an elixir, a tincture, a paste, a foam, an aerosol, a spray, a patch, a transdermal patch, via roll-on applicator microneedles and an applicator syringe. In another embodiment, the non-infection granulomatous disease includes cutaneous sarcoidosis or ocular sarcoidosis.

In another embodiment, the composition further comprises a PDE4 inhibitor selected from the group consisting of roflumilast, apremilast, crisaborole, rolipram, cilomilast, ibudilast, piclamilast and combinations thereof.

### Methods of Use

According to an aspect of the invention, the methods of this invention comprise administering a composition described herein.

According to an aspect of the invention, there is provided a method of treatment, prevention or alleviation of granuloma annulare, subcutaneous inflammation, or a non-infection granulomatous disease in a subject, wherein the method comprises administering a composition described herein. In another embodiment, the non-infection granulomatous disease includes cutaneous sarcoidosis or ocular sarcoidosis.

In some embodiments, this inventions provides a method for the treatment, prevention and/or amelioration of granuloma annulare, a non-infection granulomatous disease, or a subcutaneous inflammation, wherein the method comprises administering a dosage form comprising a therapeutically effective amount of a topical or intralesional composition comprising from about 0.25% w/w to about 10.0% w/w tapinarof and a carrier, wherein the composition is formulated in a dosage form selected a cream, a lotion, a gel, an ointment, an emulsion, a solution, a suspension, an elixir, a tincture, a paste, a foam, an aerosol, a spray, a patch, a transdermal patch, via roll-on applicator microneedles and an applicator syringe.

In another embodiment, the composition further comprises a PDE4 inhibitor selected from the group consisting of roflumilast, apremilast, crisaborole, rolipram, cilomilast, ibudilast, piclamilast and combinations thereof.

In certain embodiments, the granuloma annulare is selected from localized granuloma annulare, disseminated granuloma annulare, subcutaneous granuloma annulare or perforating granuloma annulare.

In another embodiment, the non-infection granulomatous disease includes cutaneous sarcoidosis or ocular sarcoidosis.

In other aspects of this invention, the method comprises treating, preventing or alleviating a subject afflicted with granuloma annulare. In other aspects of this invention, the method comprises treating, preventing or alleviating a subject afflicted with localized granuloma annulare. In other aspects of this invention, the method comprises treating, preventing or alleviating a subject afflicted with disseminated granuloma annulare. In other aspects of this invention, the method comprises treating, preventing or alleviating a subject afflicted with subcutaneous granuloma annulare. In other aspects of this invention, the method comprises treating, preventing or alleviating a subject afflicted with perforating granuloma annulare. In other aspects of this invention, the method comprises treating, preventing or alleviating a subject afflicted with subcutaneous inflammation. In other aspects of this invention, the method comprises treating, preventing or alleviating a subject afflicted with both granuloma annulare and subcutaneous inflammation. In other aspects of this invention, the method comprises treating, preventing or alleviating a subject afflicted with cutaneous sarcoidosis. In other aspects of this invention, the method comprises treating, preventing or alleviating a subject afflicted with ocular sarcoidosis.

In other aspects, the subcutaneous inflammation is selected from hidradenitis suppurativa, prurigo nodularis and panniculitis.

### Hidradenitis Suppurativa (HS)

Hidradenitis suppurativa (HS), also known as acne inversa, is a long-term chronic skin disease whose present treatment options are often unsatisfactory. HS has a profound effect on patient's quality of life (QoL). Alavi A. et al., reviewed QoL aspects of this disease in an article titled “Quality-of-Life Impairment in Patients with Hidradenitis Suppurativa” (Am J Clin Dermatol, 2014, vol. 15. No. 6).

The clinical picture of HS includes solitary nodules, diffuse, painful abscesses, malodorous drainage, sinus tract formation and scarring.

The exact cause of hidradenitis suppurativa is still unclear, but it is believed that the underlying mechanism involves dysfunction of the apocrine sweat glands or hair follicles.

### Prurigo Nodularis

Prurigo nodularis is a skin disease characterised by pruritic (itchy) nodules which usually appear on the arms or legs. Patients often present with multiple excoriated lesions caused by scratching. Prurigo nodularis is very hard to treat, but current therapies include steroids, vitamins, cryosurgery, thalidomide and UVB light.

### Panniculitis

Panniculitis is a group of skin conditions that involve inflammation of subcutaneous fat, cause painful bumps, or nodules under the skin—often on the legs and feet. These bumps create inflammation in the fat layer under the skin. This layer is called the panniculus, or subcutaneous fat layer. It's the type of fat that provides insulation and helps regulate the body temperature. There are many different types of panniculitis. The type of Panniculitis depends on the area of the fat cell the inflammation is in.

In some aspects, side effects following the treatment with tapinarof composition include burning, itching, inflammation, or peeling are avoided by using the methods and compositions of this invention.

According to an aspect of the invention, there is provided a method of treatment, prevention or alleviation of subcutaneous inflammation in a subject. In other aspects of this invention, the method comprises treating, preventing or alleviating a subject afflicted with hidradenitis suppurativa. In other aspects of this invention, the method comprises treating, preventing or alleviating a subject afflicted with prurigo nodularis. In other aspects of this invention, the method comprises treating, preventing or alleviating a subject afflicted with panniculitis.

### Sarcoidosis (Cutaneous Sarcoidosis, Ocular Sarcoidosis)

According to an aspect of the invention, there is provided a method of treatment, prevention or alleviation of sarcoidosis in a subject by administering a composition of this invention.

In other aspects the sarcoidosis comprises cutaneous sarcoidosis, or ocular sarcoidosis. In other aspects of this invention, the method comprises treating, preventing or alleviating a subject afflicted with cutaneous sarcoidosis, by administering a composition described herein.

In other aspects of this invention, the method comprises treating, preventing or alleviating a subject afflicted with ocular sarcoidosis, by administering a composition described herein.

Sarcoidosis is an inflammatory disease of unknown cause and highly variable epidemiology. It can affect virtually any organ system in the human body. The skin is second only to the lungs in being the most commonly affected part of the body and may be involved in 25% to 30% of cases. Other organs that are often affected include the skin, lungs, lymph nodes, and eyes but can affect virtually any organ heart, the eyes, the major nerves of the head and neck, the spinal cord, and the brain, with cardiac and pulmonary involvement being the most common causes of death.

Among people with sarcoidosis, the incidence of cutaneous sarcoidosis is extremely variable, ranging from 5% to 90%. Sarcoidosis manifests as lesions in different parts of the body that show signs of a particular type of inflammatory reaction known as non-caseating granulomas.

Granuloma annulare (GA) and sarcoidosis are two diseases of unknown cause which involve the skin and whose basic pathology is a mononuclear histiocytic cellular reaction. Biopsy plays the major role in the diagnosis of both diseases, and no other routine laboratory test for either disease is currently available.

Sarcoidosis is generally considered to be an allergic or immune granuloma with inconstant defects in cell-mediated immunity (Broom & MacLaurin, 1973, Clin. Exp. Immunol., 15, 355,).

Sarcoidosis is a global disease but is more common in Nordic and African American populations.

The prevalence of ocular involvement in systemic sarcoidosis ranges from 12%-76% with ocular involvement being the presenting symptom in 30-40%. The most common types of ocular involvement are uveitis and conjunctival nodules.

Cutaneous Sarcoidosis forms includes: Papular, Maculopapular, Plaque Annular, Lupus pernio, Subcataneous nodules, scar, angiolupoid, hypopigmented, atrophic, psoriasiform, verrucous, necrobiosis, ichthyosiform, ichthyosiform, erythrodermic, morpheaform, oral cavity, scalp, nail or genital.

In other aspects of this invention, the method comprises treating, preventing or alleviating a subject afflicted with cutaneous sarcoidosis, by administering a composition described herein, wherein the cutaneous sarcoidosis consists of at least one form selected from: Papular, Maculopapular, Plaque Annular, Lupus pernio, Subcataneous nodules, scar, angiolupoid, hypopigmented, atrophic, psoriasiform, verrucous, necrobiosis, ichthyosiform, ichthyosiform, erythrodermic, morpheaform, oral cavity, scalp, nail and genital.

In other aspects of this invention, the method comprises treating, preventing or alleviating a subject afflicted with ocular sarcoidosis, by administering a composition described herein, wherein the ocular sarcoidosis involves uveitis, conjunctival nodules or combination thereof.

According to an aspect of the invention, there is provided a method of treatment, prevention or alleviation of granuloma annulare, a non-infection granulomatous disease (such as cutaneous sarcoidosis, ocular sarcoidosis), or a subcutaneous inflammation, the method comprises administration to a subject in need thereof from about 0.25% w/w to about 10.0% w/w tapinarof composition, suitable for topical or intralesional administration.

According to an aspect of the invention, there is provided a method of treatment, prevention or alleviation of granuloma annulare, a non-infection granulomatous disease, or a subcutaneous inflammation, the method comprises administration to a subject in need thereof from about 0.25% w/w to about 5.0% w/w tapinarof composition, suitable for topical or intralesional administration. In another embodiment, the non-infection granulomatous disease includes cutaneous sarcoidosis or ocular sarcoidosis.

According to an aspect of the invention, there is provided a method of treatment, prevention or alleviation of granuloma annulare, a non-infection granulomatous disease, or a subcutaneous inflammation, the method comprises administration to a subject in need thereof a composition comprising from about 0.25% w/w to about 10.0% w/w tapinarof, from about 0.25% w/w to about 3% w/w of a PDE4 inhibitor and a carrier suitable for topical or intralesional administration. In certain embodiments, the PDE4 inhibitor is roflumilast. In another embodiment, the non-infection granulomatous disease includes cutaneous sarcoidosis or ocular sarcoidosis.

According to an aspect of the invention, there is provided a method of treatment, prevention or alleviation of granuloma annulare, a non-infection granulomatous disease, or a subcutaneous inflammation, the method comprises administration to a subject in need thereof a composition comprising from about 0.25% w/w to about 5% w/w tapinarof, from about 0.25% w/w to about 3% w/w of a PDE4 inhibitor and a carrier suitable for topical or intralesional administration. In certain embodiments, the PDE4 inhibitor is roflumilast. In another embodiment, the non-infection granulomatous disease includes cutaneous sarcoidosis or ocular sarcoidosis.

In some embodiments, this invention provides a method of treatment, prevention or alleviation of granuloma annulare, a non-infection granulomatous disease, or a subcutaneous inflammation, comprising administering to a subject in need thereof a therapeutically effective amount of a topical or intralesional composition of this invention. In another embodiment, the composition is administered at least once daily. In another embodiment, the composition is administered once daily or twice daily. In another embodiment, the composition of this invention is administered for a period between two weeks to six weeks up to six months or until remission. In another embodiment, the non-infection granulomatous disease includes cutaneous sarcoidosis or ocular sarcoidosis.

In some embodiments, this invention provides a method of treatment, prevention or alleviation of granuloma annulare, a non-infection granulomatous disease, or a subcutaneous inflammation, comprising administering to a subject in need thereof a therapeutically effective amount of a topical or intralesional a first composition comprising from about 0.25% w/w to about 10.0% w/w tapinarof and a second composition comprising from about 0.25% w/w to about 3% w/w at least one PDE4 inhibitor, wherein the first composition and second composition are administered concurrently or sequentially, at least once a day until remission. In another embodiment, the first composition and the second composition are administered for a period between two weeks to six weeks, up to six months. In certain embodiments, the PDE4 inhibitor is roflumilast. In another embodiment, the non-infection granulomatous disease includes cutaneous sarcoidosis or ocular sarcoidosis.

In another embodiment, the composition is a gel. In another embodiment, the gel composition comprises 2% w/w tapinarof. In another embodiment, the composition is a lotion.

According to an aspect of the invention, there is provided a method of treatment, prevention or alleviation of granuloma annulare, a non-infection granulomatous disease (such as cutaneous sarcoidosis, ocular sarcoidosis), or a subcutaneous inflammation.

### Regimen

Therapeutically effective concentrations of tapinarof in the compositions of this invention for treatment, prevention or alleviation of granuloma annulare, a non-infection granulomatous disease, or a subcutaneous inflammation are determined by empirical methods known in the art. In another embodiment, the non-infection granulomatous disease includes cutaneous sarcoidosis or ocular sarcoidosis.

In some embodiments, this invention provides a regimen for treating granuloma annulare, a non-infection granulomatous disease, or a subcutaneous inflammation comprises administering a therapeutically effective amount of a composition of this invention at least once daily. In another embodiment, the administration is once daily or twice daily to a patient in need thereof until remission. In another embodiment, the non-infection granulomatous disease includes cutaneous sarcoidosis or ocular sarcoidosis.

In another embodiment, the composition is administered for a period of between two weeks to six weeks up to six months or until remission. In another embodiment, the composition is administered for a period of between two weeks to six weeks. In another embodiment, the composition is administered for a period of between two weeks to four weeks. In another embodiment, the composition is administered for a period of between one week to four weeks. In some embodiments, the composition is administered for a period longer than six weeks.

### Kit

In some embodiment, this invention provides a kit comprising one or more dosage forms of this invention and instructions for use.

Kits are provided, containing the composition of this invention, optionally including instructions for administration.

The compositions provided herein can be packaged as articles of manufacture containing packaging material, a composition provided herein, and a label that indicates that the composition is for treatment, prevention or alleviation of granuloma annulare, a non-infection granulomatous disease, or subcutaneous inflammation and is formulated for topical or intralesional delivery. In another embodiment, the non-infection granulomatous disease includes cutaneous sarcoidosis or ocular sarcoidosis.

The articles of manufacture provided herein contain packaging materials. Packaging materials for use in packaging pharmaceutical products are well known to those of skill in the art. Examples of pharmaceutical packaging materials include, but are not limited to bottles, tubes, containers, application syringes and any packaging material suitable for a selected formulation and intended mode of administration and treatment.

### Definitions

Unless defined otherwise, all technical and scientific terms used herein have the same meaning as is commonly understood by one of skill in the art to which the invention pertains. In case of conflict, the specification, including definitions, takes precedence. All patents, patent applications, published applications, articles, publications and other published materials referred to throughout the entire disclosure herein, unless noted otherwise, are incorporated by reference in their entirety.

As used herein, the indefinite articles “a” and “an” mean “at least one” or “one or more” unless the context clearly dictates otherwise.

As used herein, the term “treating” or” treatment” includes curing a condition, treating a condition, preventing a condition, treating symptoms of a condition, curing symptoms of a condition, ameliorating symptoms of a condition, treating effects of a condition, ameliorating effects of a condition, and preventing results of a condition.

As used herein, the terms “pharmaceutically active agent” or “active agent” or “active pharmaceutical ingredient” or “API” are interchangeable and mean the ingredient is a pharmaceutical drug which is biological active and is regulatory approved or approvable as such.

As used herein, a “pharmaceutical composition” refers to a composition comprising one or more active ingredients with other components such as pharmaceutically acceptable ingredients or excipients.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “10%” is intended to mean “about 10%”.

As used herein, numerical ranges preceded by the term “about” should not be considered to be limited to the recited range. Rather, numerical ranges preceded by the term “about” should be understood to include a range accepted by those skilled in the art for any given element in formulations according to the present invention.

As used herein, when a numerical value is preceded by the term “about”, the term “about” is intended to indicate +/−10%.

The terms “comprise”, “comprising”, “includes”, “including”, “having” and their conjugates mean “including but not limited to”.

The term “consisting of” means “including and limited to”.

The term “consisting essentially of” means that the composition, method formulation may include additional ingredients, steps and/or parts, but only if the additional ingredients, steps and/or parts do not materially alter the basic and novel characteristics of the claimed composition, method or structure.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

### Example 1: Treatment of Granuloma Annulare Using 1% Tapinarof

A 71-years-old woman who was more than 5 years diagnosed with Generalized Granuloma Annulare was treated during that period with cycles of topical corticosteroids, moderate to potent class, without any improvement.

The woman was treated with 1% tapinarof lotion (as described in Example 2)—as an experimental phase, by applying topically only on one lesion twice a day—to verify its effect.

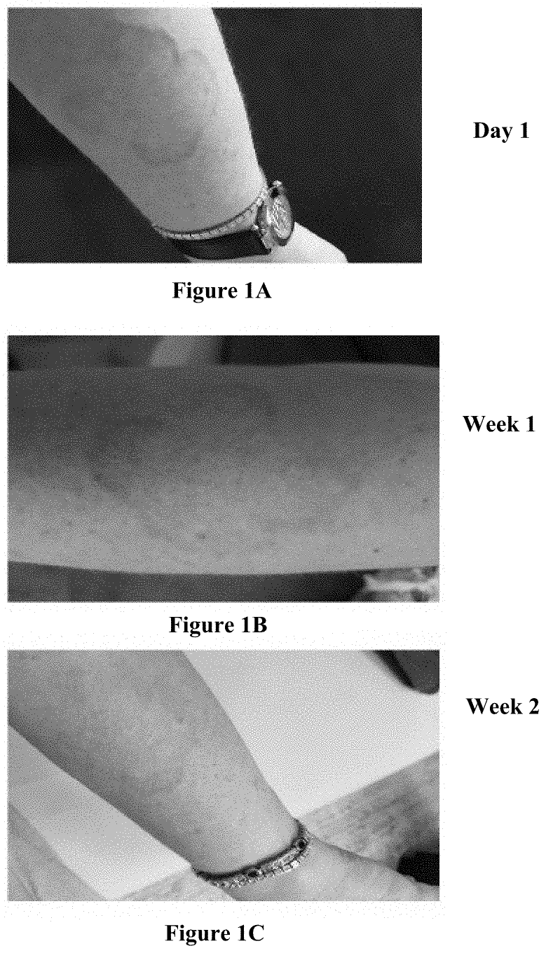

After two weeks a clear improvement was observed especially in the inflammatory infiltrate of elevated rolled border. There was also an improvement in the telengiectatic vessels aspect of the central patch. (FIG. 1).

No undesirable side effects such as burning, itching or peeling were noted. The treatment was continued for another 4 weeks.

Following the 4 weeks treatment, a rebound effect appeared with erythema, pruritus and folliculitis (in tapinarof treatments it is known that 10% can have folliculitis as a side effect. An anti-folliculitis product can be used in order to overcome the side effect).

The side effect disappeared after 5-6 days using Elocom (an anti-inflammatory agent).

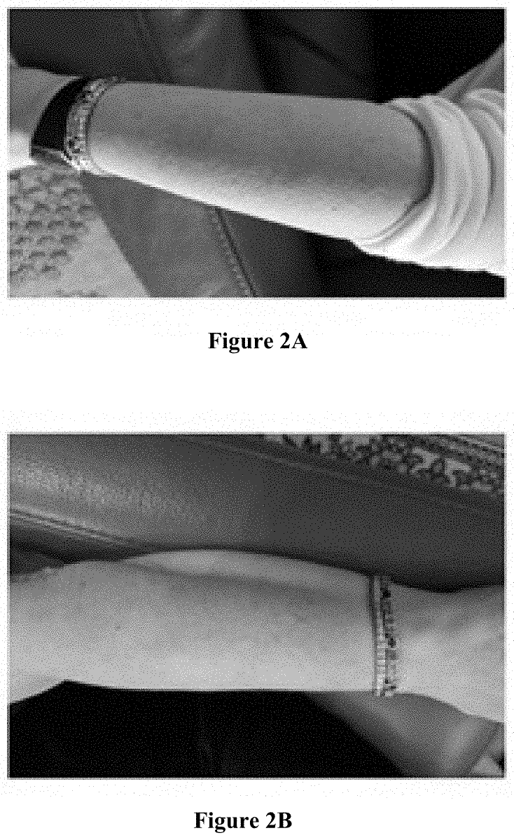

In the right side of FIG. 2, a light peeling is observed, and no trace of Granuloma, and no pruritus are observed.

### Example 2: Preparation of Tapinarof, 1% Lotion

| Raw Material (compendial Name) | % w/w | Function |
|---|---|---|
| Tapinarof 98% | 1.0 | Active |
| Castor oil | 4.0 | Emollient |
| Mineral oil light | 4.0 | Emollient |
| Diethylene glycol monoethyl ether | 5.5 | Solvent, penetration |
| (Transcutol) | | agent |
| Dimethyl Sulfoxide | 5.5 | Solvent, penetration |
| | | agent |
| Sorbitan Monooleate (Span 80) | 0.1 | Surfactant |
| Propylene glycol | 10.0 | Solvent |
| Disodium Edetate (EDTA) | 0.1 | Chelating agent |
| Carbomer Copolymer Type B | 0.4 | Surfactant |
| Pemulen ®TR-1 | | |
| Carbomer Homopolymer Type A | 0.6 | Gelling |
| Carbopol ®981 | | |
| Purified water | 64.00 | Continuous phase |
| Benzoic Acid | 0.25 | Preservative |
| BHT | 0.1 | Anti-oxidant |
| Citric Acid | 0.1 | Buffer |
| Sodium Citrate | 0.2 | Buffer |
| Sodium hydroxide pellets | For pH | pH adjustment |
| | adjustment | |
| Purified water | q.s. to 100 | Continuous phase |

### Water Phase

Into a glass beaker water and Benzoic Acid were added. The beaker was placed inside a hot water bath adjusted to 60° C. and the mixture was mixed with a magnetic stirrer until a clear solution free from particles was obtained. Then EDTA, Citric Acid and Sodium Citrate were added. The mixing was continued until a clear solution was obtained. The solution was cooled down to room temperature. Then, the pH was slowly adjusted to pH 6.0 with NaOH 20%.

### Oil Phase

In a separate glass beaker Mineral oil light, castor oil, span 80 and BHT were weighed. The beaker was placed inside a hot water bath adjusted to 60° C. and the mixture was mixed with a magnetic stirrer until a uniform solution was obtained. Then Carbopol®981 and Pemulen®TR-1 were slowly added and the mixing was continued until a homogenous mixture was obtained. The mixture was cooled down to room temperature.

### Active Phase

Into a separate glass beaker Propylene Glycol, Transcutol and DMSO were weighed. The mixture was mixed with a magnetic stirrer until a uniform homogenous solution was obtained. The beaker was covered with an aluminum foil and placed in a yellow light hood. Tapinarof was slowly added, and the mixing was continued for about 1 h until a clear solution free from particles was obtained.

The oil phase was slowly added to the water phase while homogenizing for about 5 minutes, until there were no lumps. Then, the active phase was slowly added to the Water+Oil phase while homogenizing for about 5 minutes.

Water was added for batch completion, and final pH was measured to conform it is around pH 5.

### Example 3: Tapinarof, 2% Hydrogel

| Target | Raw Material (compendial Name) | % w/w |
|---|---|---|
| API | Tapinarof | 2.00 |
| Continuous phase | Purified water | 50.0 |
| Surfactant | Polysorbate 20 | 3.00 |
| Wetting agent | Glycerin | 5.00 |
| Gelling agent | Natrosol ™ hydroxyethyl cellulose | 1.50 |
| Solvent | Diethyl sebacate | 10.00 |
| Penetration enhancer | Isopropyl myristate | 25.00 |
| Preservative | Phenoxyethanol | 0.15 |
| Antioxidant | Propyl gallate | 0.3 |
| q.s. to 100 | Purified water | q.s. to 100 |

### Water Phase

In a glass beaker, water was weighed, then Tween 20 was added, and the mixture was mixed with a mechanical mixter until a clear solution was obtained. Then, natrosol was added gradually while mixing continues until the swollen particles dissolve to form viscous smooth mixture.

### Active Phase:

Into a separate glass beaker glycerin, diethyl sebacate, isopropyl myristate, phenoxyethanol and propyl gallate were weighed

The beaker was placed inside a hot water bath adjusted to 65° C. The mixture was mixed with a magnetic stirrer until a homogenous solution was obtained. The mixture was cooled down to room temperature. The beaker was covered with an aluminum foil and placed inside yellow light hood. Then, tapinarof was added slowly to mixture, and the mixing was continued for at least 1 hour until a clear solution free from particles was obtained.

The Active phase was slowly added to the water phase while homogenizing, until a homogenous gel was formed fee from lumps.

### Example 4: Tapinarof, 2% Hydrogel

| Target | Raw Material (compendial Name) | % w/w |
|---|---|---|
| API | Tapinarof | 2.00 |
| Continuous phase | Purified water | 50.0 |
| Surfactant | Polysorbate 60 | 4.0 |
| Wetting agent | PEG 400 | 5.00 |
| Gelling agent | Carbopol ®980 polymer | 1.50 |
| Solvent | Isosorbid dimethyl ether | 15.00 |
| Penetration enhancer | Oleyl alcohol | 10.00 |
| Preservative | Chlorocresol | 0.2 |
| Antioxidant | Ascorbic acid | 0.30 |
| pH adjustment | Sodium hydroxide pellets | For pH |
| | | adjustment |
| q.s. to 100 | Purified water | q.s. to 100 |

### Gelling Phase

In a glass beaker, water was weighed, then polysorbate 60 and ascorbic acid were added, and the mixture was mixed with a magnetic stirrer until a clear solution was obtained.

Into a separate glass beaker PEG 400 was weighed followed by addition of Carbopol, the mixture was mixed with a spatula until all powder was dispersed and homogenous paste was formed.

The water solution was added to the paste and left to rest for about 2 h at room temperature in order to enable sufficient swelling. After the swelling, the pH of the gelling phase was adjusted to pH 4.5-5.0 with NaOH 20%.

### Active Phase

In a separate glass beaker isosorbide dimethyl ether, and oleyl alcohol were weighed. The mixture was mixed with a magnetic stirrer until a homogenous clear solution was obtained. The beaker was placed inside a hot water bath adjusted to 65° C. chlorocresol was added and the mixing was continued until all powder was dissolved. The mixture was cooled down to room temperature. The beaker was covered with an aluminum foil and placed inside yellow light hood. Then, tapinarof was added slowly to the mixture, and the mixing was continued for at least 1 hour until a clear solution free from particles was obtained.

The Active phase was slowly added to the gelling phase while homogenizing, until a homogenous gel was formed. Water was added for batch completion, and final pH was measured to confirm it is around pH 5.

### Example 5: Tapinarof, 2% Hydrogel

| Target | Raw Material (compendial Name) | % w/w |
|---|---|---|
| API | Tapinarof | 2.00 |
| Continuous phase | Purified water | 45.0 |
| Surfactant | Polysorbate 80 | 5.0 |
| Wetting agent | PEG 400 | 5.00 |
| Gelling agent | Carbopol ®980 polymer | 1.20 |
| Solvent | Propylene glycol | 10.00 |
| Penetration enhancer | Oleic acid | 23.00 |
| Preservative | Methylparaben | 0.80 |
| Antioxidant | Butylated hydroxytoluene | 0.10 |
| pH adjustment | Sodium hydroxide pellets | For pH |
| | | adjustment |
| q.s. to 100 | Purified water | q.s. to 100 |

### Gelling Phase

In a glass beaker, water was weighed, then polysorbate 80 was added, and the mixture was mixed with a magnetic stirrer until a clear solution was obtained.

Into a separate glass beaker PEG 400 was weighed followed by addition of Carbopol, the mixture was mixed with a spatula until all powder was dispersed and homogenous paste was formed.

The water solution was added to the paste and left to rest for about 2 h at room temperature in order to enable sufficient swelling. After the swelling, the pH of the gelling phase was adjusted to pH 4.5-5.0 with NaOH 20%.

### Active Phase

In a separate glass beaker propylene glycol, and oleic acid were weighed. The mixture was mixed with a magnetic stirrer until a homogenous clear solution was obtained. The beaker was placed inside a hot water bath adjusted to 65° C. methyl paraben and BHT were added and the mixing was continued until all powder was dissolved. The mixture was cooled down to room temperature. The beaker was covered with an aluminum foil and placed inside yellow light hood. Then, tapinarof was added slowly to the mixture, and the mixing was continued for at least 1 hour until a clear solution free from particles was obtained.

The Active phase was slowly added to the gelling phase while homogenizing, until a homogenous gel was formed. Water was added for batch completion, and final pH was measured to confirm it is around pH 5.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A method of treatment, prevention or alleviation of granuloma annulare, a non-infection granulomatous disease, or a subcutaneous inflammation, comprising administering to a subject in need thereof a therapeutically effective amount of a topical or intralesional composition comprising from about 0.25% w/w to about 10.0% w/w tapinarof and a carrier.

2. The method of claim 1, wherein the composition comprises 1% or 2% w/w tapinarof.

3. The method of claim 1, wherein the purity of the tapinarof is greater than 97%.

4. The method of claim 1, wherein the subcutaneous inflammation is selected from the group consisting of hidradenitis suppurativa, prurigo nodularis and panniculitis.

5. The method of claim 1, wherein the non-infection granulomatous disease is selected from the group consisting of cutaneous sarcoidosis and ocular sarcoidosis.

6. The method of claim 1, wherein the composition further comprises a PDE4 inhibitor.

7. The method of claim 6, wherein the PDE4 inhibitor is selected from the group consisting of roflumilast, apremilast, crisaborole, rolipram, cilomilast, ibudilast, piclamilast and combinations thereof.

8. The method of claim 7, wherein the PDE4 inhibitor is roflumilast.

9. The method of claim 6, wherein the composition comprises from about 0.25% w/w to about 10.0% w/w tapinarof and from about 0.25% w/w to about 3% w/w at least one PDE4 inhibitor.

10. The method of 1, wherein the composition is formulated as a gel, a cream, a foam or as a lotion.

11. The method of claim 10, wherein the composition is a gel, comprising a surfactant, a solvent, a wetting agent, a gelling agent, a penetration enhancer, a preservative and an anti-oxidant.

12. The method of claim 10, wherein the composition is a lotion, comprising a surfactant, an emollient, a solvent, a gelling agent, a penetration enhancer, a chelating agent, a preservative, a buffer and an anti-oxidant.

13. The method of claim 1, wherein the method comprises administering at least once daily a therapeutically effective amount of the composition.

14. The method of claim 13, wherein the method comprises administering once daily or twice daily a therapeutically effective amount of the composition.

15. The method of claim 1, wherein the method comprises administering the composition at least once daily for a period between two weeks to six weeks, up to six months or until remission.

16. A method of treatment, prevention or alleviation of granuloma annulare, a non-infection granulomatous disease, or a subcutaneous inflammation, comprising administering to a subject in need thereof a therapeutically effective amount of a topical or intralesional a first composition comprising from about 0.25% w/w to about 10.0% w/w tapinarof and a second composition comprising from about 0.25% w/w to about 3% w/w PDE4 inhibitor, wherein the first composition and second composition are administered concurrently or sequentially, at least once a day until remission.

17. The method of claim 16, wherein the PDE4 inhibitor is roflumilast.

18. The method of claim 16, wherein the non-infection granulomatous disease is selected from the group consisting of cutaneous sarcoidosis and ocular sarcoidosis.

19. A dosage form for the treatment, prevention and/or amelioration of granuloma annulare, a non-infection granulomatous disease, or a subcutaneous inflammation, wherein the dosage form comprising a therapeutically effective amount of a topical or intralesional composition comprising from about 0.25% w/w to about 10.0% w/w tapinarof and a carrier, wherein the composition is formulated in a dosage form selected from a cream, a lotion, a gel, an ointment, an emulsion, a solution, a suspension, an elixir, a tincture, a paste, a foam, an aerosol, a spray, a patch, a transdermal patch, via roll-on applicator, microneedles and an applicator syringe.

20. The dosage form of claim 19, wherein the composition further comprises a PDE4 inhibitor selected from the group consisting of roflumilast, apremilast, crisaborole, rolipram, cilomilast, ibudilast, piclamilast and combinations thereof.

21. The dosage form of claim 19, wherein the non-infection granulomatous disease is selected from the group consisting of cutaneous sarcoidosis and ocular sarcoidosis

22. A kit comprising one or more dosage forms of claim 19 and instructions for use.
